(19) United States
(12) Patent Application Publication
(10) Pub. No.: US 2016/0262923 A1
(43) Pub. Date: Sep. 15, 2016

(54) INTRAUTERINE DELIVERY SYSTEM

(71) Applicant: BAYER OY, Turku (FI)

(72) Inventors: Manja AHOLA, Piikkiö (FI); Christine TALLING, Turku (FI); Bernhard LINDENTHAL, Berlin (DE); Ulrike FUHRMANN, Berlin (DE); Lüder M. FELS, Bad Freienwalde / OT Schi (DE); Katja PRELLE, Ratingen (DE); Norbert SCHMEES, Berlin (DE)

(73) Assignee: Bayer Oy, Turku (FI)

(21) Appl. No.: 15/030,046

(22) PCT Filed: Oct. 14, 2014
(86) PCT No.: PCT/EP2014/071990

§ 371 Date: Apr. 15, 2016

Related U.S. Application Data
(60) Provisional application No. 61/893,083, filed on Oct. 18, 2013.

(30) Foreign Application Priority Data
Oct. 21, 2013 (EP) 13397533.4

(51) Int. Cl. A61F 6/14; A61M 31/00; A61K 31/567; A61K 47/34; A61K 9/00; A61K 31/58

(52) U.S. Cl. CPC A61F 6/144; A61K 9/0039; A61K 31/58; A61K 31/567; A61K 47/34; A61M 31/002

(57) ABSTRACT

The present invention relates to an improved method of contraception which addresses the problems of initial bleeding and spotting associated with the use of intrauterine delivery systems, and to an improved intrauterine delivery system.

### CROSS-REFERENCE TO RELATED APPLICATIONS

This application is a U.S. national phase application of International Patent Application No. PCT/EP2014/071990, filed Oct. 14, 2014 and titled “Intrauterine Delivery System,” which claims priority to both U.S. Provisional Patent Application No. 61/893,083, filed Oct. 18, 2013 and titled “Intrauterine Delivery System,” and European Patent Application No. 13397533.4, filed Oct. 21, 2013 and titled “Intrauterine Delivery System,” the contents of each of which are incorporated herein by reference in their entirety.

The present invention relates to an improved method of contraception which addresses the problems of initial bleeding and spotting associated with the use of intrauterine delivery systems, and to an improved intrauterine delivery system.

Bleeding disorders are one of the most frequent gynecological problems. The causes of bleeding disorders, and their frequency in particular, vary depending on the age of the woman affected. A levonorgestrel-releasing intrauterine system (LNG-IUS, for example Mirena®) has been shown to be effective as such in the treatment of heavy menstrual blood losses. This product is described in, inter alia, EP 0652738 B1 and EP 0652737 B1.

Mirena® is a systemic hormonal contraceptive that provides an effective method for long term contraception and complete reversibility, and has an excellent tolerability record. The local release of levonorgestrel (active ingredient in Mirena®) within the endometrial cavity results in strong suppression of endometrial growth as the endometrium becomes insensitive to ovarian estradiol. The endometrial suppression is the reason for a reduction in the duration and quantity of menstrual bleeding and alleviates dysmenorrhea.

Although the contraceptive effect of Mirena® is mainly a result of a local effect, the comparatively high systemic stability of levonorgestrel means that Mirena® also exhibits plasma levels of active ingredient of on average about 206 pg/ml¹. Although this value is below that of orally administered levonorgestrel-containing contraceptives, it is still high enough for it to inhibit ovulation in about 20% of users in the first year of use and for it to be able to cause the known systemic adverse effects, for example acne, depressed moods, chest pain or reduced libido². ¹ See information sheet Mirena March 2011—DE/9² Lähteenmäki P. et al. Steroids 2000 65: 693-697

However, during the first months of use of an IUS or IUD (Intrauterine Delivery Device), irregularity in vaginal bleeding patterns is the most common clinical side effect^{3,4}. The irregularities may include an increase in the menstrual blood loss at cyclical periods, increased duration of bleeding at periods, and intermenstrual bleeding and spotting. ³ Guillebaud 1976 et al. and Shaw et al 1980Pedron 1995, Adv Contra Deliv Syst Vol 11,245

With LNG-IUS, users experience undesirable bleeding, particularly during the first 3 to 6 cycles after insertion. Only some of the users experience complete amenorrhea even after long-term usage, and users often report occasional bleeding incidents that are irregular and unpredictable, especially during the first few months of use. Irregular bleeding is thus a common initial complaint among users and often a reason for discontinuing the use of the IUS. It may take up to six months for the reduction of heavy menstrual bleeding (HMB) to reach maximum effect. Therefore there is still a need for an intrauterine delivery system, the use of which would offer an improved and safe method of contraception and address the initial bleeding problems by suppressing abnormal and/or irregular bleeding especially during the first three to six months of use.

In some implementations, the specification describes an improved method for contraception and for preventing or suppressing initial bleeding during the first months of use of an intrauterine delivery system by using an intrauterine delivery system comprising two reservoirs which comprise progestogen or a drug having progestogenic activity and have different release kinetics over a prolonged period of time.

In some implementations, the specification describes an intrauterine delivery system comprising two reservoirs which comprise progestogen or a drug having progestogenic activity and release the same at constant, predefined rates which are different from the two reservoirs.

In some implementations, the specification describes a contraceptive intrauterine system which addresses the initial bleeding problems but which provides the desired contraceptive effect with the benefit of lower systemic side effects and thus further improved tolerability.

In some implementations, the specification describes the use of an intrauterine delivery system which comprises a body construction and two reservoirs both comprising a core and a membrane encasing at least part of the core, the core and the membrane essentially consisting of the same or a different polymer composition, whereby it is preferred that the core and the membrane are different polymers, wherein the reservoirs comprise a progestogen or a drug having progestogenic activity and have different release kinetics.

### BRIEF DESCRIPTION OF THE DRAWINGS

FIG. 1 illustrates release rates obtained from the reservoirs separately and as a combined system.

FIG. 2 illustrates bleeding results for each animal as a function of different drug dosages.

FIG. 3 illustrates bleeding days for each animal as a function of different drug dosages.

FIG. 4 illustrates dose-dependent weight gain as a function of different drug dosages.

FIG. 5 illustrates luteinizing hormone levels as a function of different drug dosages.

FIG. 6 illustrates fold induction as a function of different drug dosages.

FIG. 7 illustrates example intrauterine systems.

### DETAILED DESCRIPTION

Two reservoirs in the context of this invention means that the IUS contains two or more reservoirs releasing the active substance with two different release kinetics. Thus a variant of the intrauterine system could contain e.g. three reservoirs on each arm of the T-frame of the IUS, whereby two of these three reservoirs have an identical release and the 3^{rd} reservoir has a different release kinetic. E.g. the reservoir with the slow release could be mounted on the vertical stem of the T-frame and the two reservoirs with the faster release kinetic could be mounted on the horizontal arms of the T-frame.

The reservoirs comprise a core and a membrane encasing at least part of the core. The core comprises a polymer composition, that is, the core is a polymer matrix wherein the therapeutically active substance or substances are dispersed.

The release rates from the two reservoirs can be controlled by the membrane or by the membrane together with the core. The membrane may cover the whole reservoir or cover only a part of the system, for example one segment of the core.

The release rate can be influenced via selection of a polymer or a combination of polymers. The higher the amount of fluoro-modified polysiloxanes (PTFPMS) in the membrane, the lower and more constant the release rate is. If a low and constant release rate is desired, a typical PTFPMS/PDMS ratio is in the range in wt % of 100/0-10/90.

By increasing the amount of more hydrophilic polymer, like PEO-b-PDMS (polyethylene oxide block-polydimethylsiloxane), in the membrane the release can be increased. If a high release rate is desired typical PEO-b-PDMS/PDMS ratio is in the range in wt % of 95/5-0/100.

The release rate can be controlled by physical dimensions of the drug reservoir, for example, outer dimensions of the reservoir or the thickness of the release rate controlling membrane. A higher release rate can be obtained by increasing the surface area and length or by using a thinner membrane. The thicker the membrane the lower the release rate. If a high release rate, is desired typical membrane thickness is in the range of 0.15 to 0.3 mm. For a slow release rate, the desired membrane thickness is in the range of 0.3 to 0.6 mm.

The release rate can be further controlled by adjusting the silica filler content in the membrane, the higher the silica filler content in the membrane the lower the release rate.

The membrane may consist of more than one layer. The combination of different membrane layers as regards thickness or material or both gives a further possibility to control the release rates of the active agents.

Drug load in the core has a minor effect on the release rate, the higher the drug load in the core, the more constant the release is. Drug load has an influence on the duration of the drug release, the higher the load is, the longer the duration. Thus the drug loads in reservoir 1 and reservoir 2 can be different depending on the time the IUS is in use.

Polysiloxanes are known to be suitable for use as a membrane or matrix regulating the permeation rate of drugs. Polysiloxanes are physiologically inert, and a wide group of therapeutically active substances are capable of penetrating polysiloxane membranes, which also have the required strength properties.

Poly(disubstituted siloxanes) where the substituents are lower alkyl, preferably alkyl groups of 1 to 6 carbon atoms, or phenyl groups, wherein said alkyl or phenyl can be substituted or unsubstituted, are preferred. A widely used and preferred polymer of this kind is poly(dimethylsiloxane) (PDMS). Other preferred polymers are siloxane-based polymers comprising either 3,3,3 trifluoropropyl groups attached to the silicon atoms of the siloxane units (fluoro-modified polysiloxanes) or poly(alkylene oxide) groups, wherein said poly(alkylene oxide) groups are present as alkoxy-terminated grafts or blocks linked to the polysiloxane units by silicon-carbon bonds or as a mixture of these forms. Polysiloxanes and modified polysiloxane polymers are described for example in EP 0652738 B1, WO 00/29464 and WO 00/00550. Among siloxane-based polymers comprising poly(alkylene oxide) groups, polyethylene oxide block-polydimethylsiloxane copolymer (PEO-b-PDMS) is preferred.

According to the first embodiment of the invention the different release kinetics of the two reservoirs are achieved by different ratios of fluoro-modified polysiloxanes to poly(dimethyl siloxane) and/or poly(alkylene oxide) modified polysiloxanes in the membrane covering the core.

The fast initial release from reservoir 1 may according to the invention be achieved by a membrane consisting of PDMS only, a PEO-b-PDMS/PDMS elastomeric mixture, a PTFPMS/PDMS elastomeric mixture and/or (PEO-b-PDMS). The ratios of different polysiloxanes or modified polysiloxanes in the membrane of reservoir 1 may vary from 0-100%. Preferably the PEO-b-PDMS/PDMS ratio in the membrane of reservoir 1 is in the range of 95/5-0/100 (wt %). The PTFPMS/PDMS ratio in the membrane of reservoir 1 is preferably in the range of 20/80-0/100 (wt %). In a preferred embodiment the membrane of reservoir 1 is 100% PDMS.

The lower release rate from reservoir 2 may according to the invention be achieved by a membrane consisting of PDMS, PTFPMS and/or a PTFPMS/PDMS elastomeric mixture. The ratios of different polysiloxanes or modified polysiloxanes in the membrane of reservoir 2 may vary from 0-100%. Preferably the PTFPMS/PDMS ratio in the membrane of reservoir 2 is 100/0-10/90, even more preferably about 80/20 (wt %).

The membrane may cover the whole reservoir or only part of it. Preferably membrane thickness is around 0.15 to 0.6 mm.

Progestogen can be in principle any therapeutically active substance having enough progestogenic activity to achieve contraception. However, as explained in more detail below a preferred pro-gestogenic compound is levonorgestrel. A particular preferred progestogenic compound is 18-methyl-15β,16β-methylene-19-nor-20-spirox-4-en-3-one, the preparation of which is described in EP 2 038 294 B1 (example 14f). Subsequently this compound is named also as New Progestin or abbreviated as NP.

The release of progestogen from the reservoirs starts from the insertion of the intrauterine system. The release of reservoir 1 should preferably last for at least three months, or from three to six months, most preferably at least 3 months.

The daily dose released for use in humans from reservoir 1 is 10-200 μg/d, depending on the particular active ingredient. For levonorgestrel the desired release rates from reservoir 1 are 20-100 μg/d, preferably 20-50 μg/d. For 18-methyl-15β,16β-methylene-19-nor-20-spirox-4-en-3-one, the desired release rates from reservoir 1 are 10-200 μg/d, preferably 10-100 μg/d.

The release of progestogen from reservoir 2 should preferably last for from one up to ten years, or from one to five years, or preferably from three to five years. The amount of the progestogen incorporated in reservoir 2 of the delivery system varies depending on the particular progestogen and the time for which the intrauterine system is expected to provide contraception.

The daily dose released from reservoir 2 is 1-50 μg/d, preferably 1-20 μg/d, depending on the particular active ingredient. For levonorgestrel the desired release rates from reservoir 2 are 5-30 μg/d, preferably 5-20 μg/d. For 18-methyl-15β,16β-methylene-19-nor-20-spirox-4-en-3-one, the desired release rates are 1-20 μg/d, preferably 1-10 μg/d.

As in the initial phase after insertion both reservoirs contribute to the release of the active ingredient the total release of the system is the sum or the daily released doses from reservoir 1 and 2. Thus the total release in the initial phase could be in the range between 1-250 μg/d.

The amount of the progestogen incorporated in reservoirs 1 and 2 of the delivery system varies depending on the particular progestogen and the choice of the polymer material. The total load in the core may be approximately 45-55%, at most 65%, based on the weight of the core, and may be different in the cores of reservoirs 1 and 2. Preferably, the amount of progestogen or a substance having a progestogenic activity may vary from almost zero to 60 wt-%, when it is mixed into the core matrix, the preferred amount being between 5-50 wt-%. Other possible ranges of the amount of the therapeutically active agent are 0.5-60 wt-%, 5-55 wt-%, 10-50 wt-%, 25-60 wt-%, 40-50 wt-% and 5-40 wt-%.

The two reservoirs may be positioned separately on the body of the delivery system. They may be attached next to each other or may be separated from each other by a separation membrane or by an inert placebo compartment. A separation membrane or an inert placebo segment provides a further means to control the release rates from the two reservoirs.

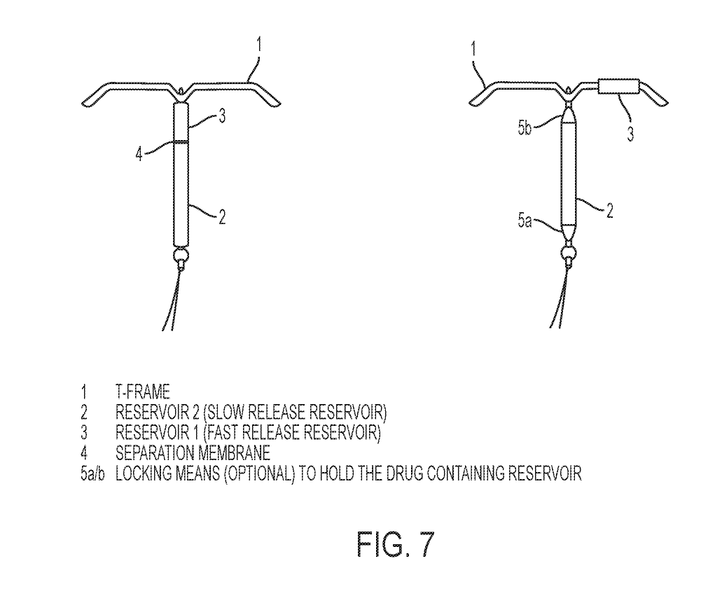

Suitable Intrauterine systems are exemplarily shown in FIG. 7. Other intrauterine systems, such as the continuous frame systems described in WO2009/122016 are similarly suitable in the context of the current invention. Reference numeral 2 in FIG. 7 refers to the slow release reservoir, reference number 3 to the fast release reservoir, reference number 1 to the T-frame, reference number 4 to a “separation” membrane, and reference number 5(a) and 5(b), respectively, to locking means which can optionally be mounted on the T-frame to hold the reservoir.

The structural integrity of the material (of the core or the membrane or both) may be enhanced by the addition of a particulate material such as silica or diatomaceous earth. Addition of silica however, has not only an impact on the mechanical integrity (strength) of the material but has also an influence on the release rate (permeability) of the membrane. Hereby the release rate decrease so more silica is added.

The core or the membrane may also comprise additional material to further adjust the release rates. Such additional material include for example complex forming agents such as cyclodextrin derivatives to adjust the initial burst of the substance to the desired level. Auxiliary substances, for example tensides, solubilisers or absorption retarders, or their mixtures may be added in order to impart the desired physical properties to the body of the delivery system.

Manufacture of intrauterine delivery systems. A person skilled in the art is familiar with the preparation of an IUS which is carried out as described, for example in EP 0 652 738 B1.

Thus the contraceptive agents are first made with a polymeric support material into a central rod (core). The active ingredient is admixed with the polymeric support material, such as PDMS as disclosed above, at a desired ratio.

After the shaping process, i.e. after curing, the core prepared in this way is surrounded in a second step by a polymer membrane, the composition of which is selected according to the invention to provide the desired release rate. As disclosed above, the desired release rate is controlled via the choice of polymer, via the thickness of the membrane, via the outer dimensions of the drug reservoir and via the silica content of the membrane and via the drug content in the core.

The membrane is applied by firstly swelling a tubing (membrane) prepared from the desired polymer in a solvent (such as cyclohexane or ethyl acetate) and then pressing the core containing the active ingredient into the still swollen tubing. After evaporation of the solvents the membrane is formed tightly around the core The ends of the tubing are then preferably also sealed by a stopper, preferably consisting of the same material as the tubing/membrane, in order to counteract “bleeding” of the active ingredient at the ends of the tubing (reservoir), which may result in a “burst effect” during use. The tubing may also be bonded with silicone in place of the stoppers.

Further alternatives to connect the membrane with the core are described in the literature, e.g. mechanical methods by applying a vacuum or pressure to the tubing membrane (an analogues method is e.g. described in EP 652 737) or via co-extrusion respectively coating extrusion or injection molding as disclosed in the handbooks of the art^{5,6}. ⁵ Chan I. Chung, Extrusion of Polymers: Theory and Practice, Hanser Publishers, Munich 2000⁶ Dominick V. Rosato, Donald V. Rosato and Marlene G. Rosato, Injection Molding Handbook, 3rd Ed, Kluwer Academic publishers, Dordrecht, 2000

Effects on (initial) bleeding and spotting: It is known that progestogen-releasing IUSs decrease the amount of menstrual bleeding compared to pre-insertion controls. The decrease in menstrual bleeding is related to the amount and/or biological potency of the steroids they release. The higher the progestational potency of the compound, the greater the decrease in menstrual bleeding. It has also been shown that there is a dose dependent effect on initial bleeding in a clinical comparison trial with different Intrauterine Systems, incl. Copper and Progestin (LNG) containing systems. The study was performed in the mid '90s by the Instituto Mexicano del Serguro Social⁷. The study showed that women treated with 8 μg/d LNG showed a greater decrease in menstrual bleeding compared to the group treated with 2 μg/d. ⁷ Pedron 1995, Adv Contra Deliv Syst Vol 11, 245

However, although a higher initial progesterone release could effectively address the problem of the initial bleeding and spotting, the upper dose is limited by the systemic side effects, which are caused by the respective progesterone, e.g. LNG. Therefore, Levonorgestrel, as used in Mirena® and investigated in the a.m. comparison trial, although suited in principle in terms of the present invention, is less advantageous in comparison to 18-methyl-15β,16β-methylene-19-nor-20-spirox-4-en-3-one [in the context of this application also referred as new progestin (NP)], which shows a low systemic stability/higher plasma clearance and higher progestional activity compared to LNG.

In some implementations, 18-methyl-15β,16β-methylene-19-nor-20-spirox-4-en-3-one is used in an intrauterine system containing reservoirs 1 and 2, wherein reservoir 1 shows a faster release than reservoir 2 and wherein reservoir 1 releases the drug essentially in the initial phase 0-6 months after insertion into the uterus of the patient and wherein reservoir 2 shows a slower release and an essentially constant release of the drug over the wearing period of up to 5 or more years.

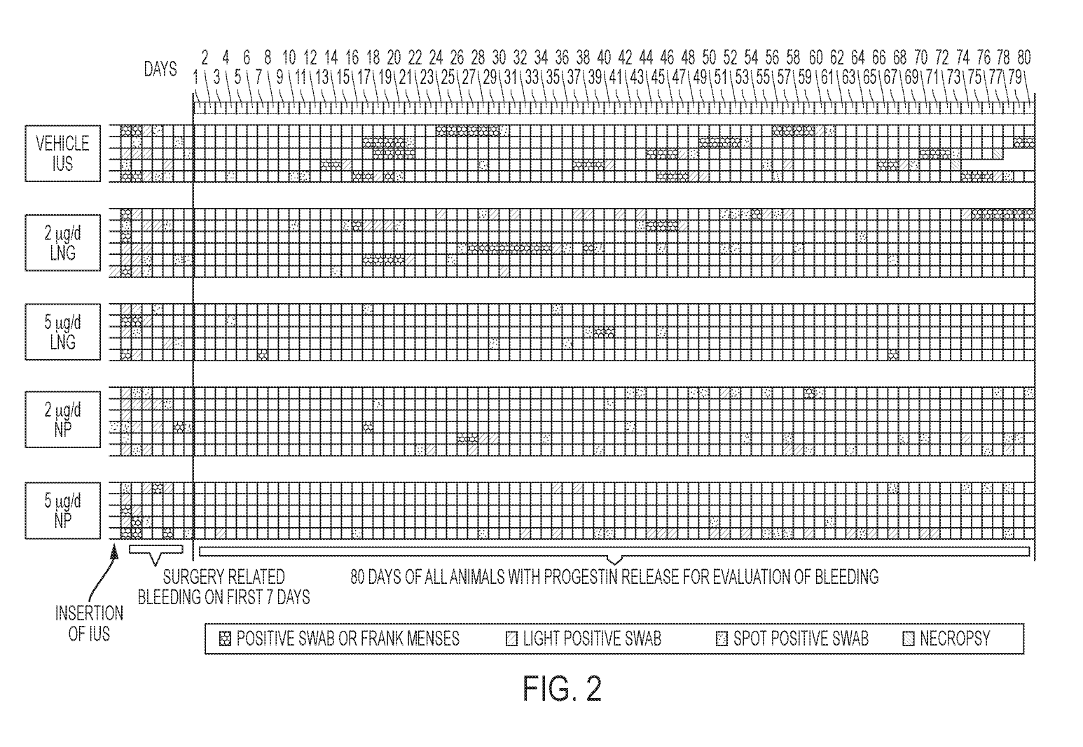

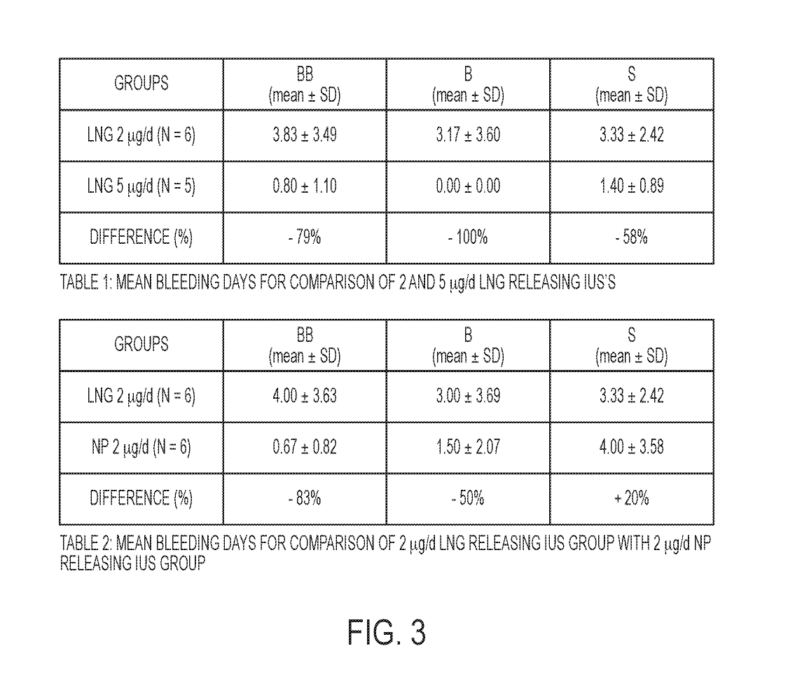

In a comparison study in monkeys with a 5 arm comparison vehicle vs. LNG vs. 18-methyl-15β,16β-methylene-19-nor-20-spirox-4-en-3-one in two dose groups (release rate of 2 μg/d and 5 μg/d for each progestin) a dose dependency of bleeding days for LNG was confirmed. Also for the NP a dose dependency was proven. In summary the number of bleeding days in the 2 μg/d LNG release group was 24/6 test animals, whereby only 4 bleeding days in 5 test animals treated with 5 μg/d LNG occurred. A similar number of bleeding days have been measured in the animal group treated with only 2 μg/d NP. The total number of bleeding days in this group was 4 (in 6 test animals). No bleeding was determined in the group treated with 5 μg/d of the NP. For further details, see also example 3 and FIGS. 2 and 3 (tables 1 and 2).

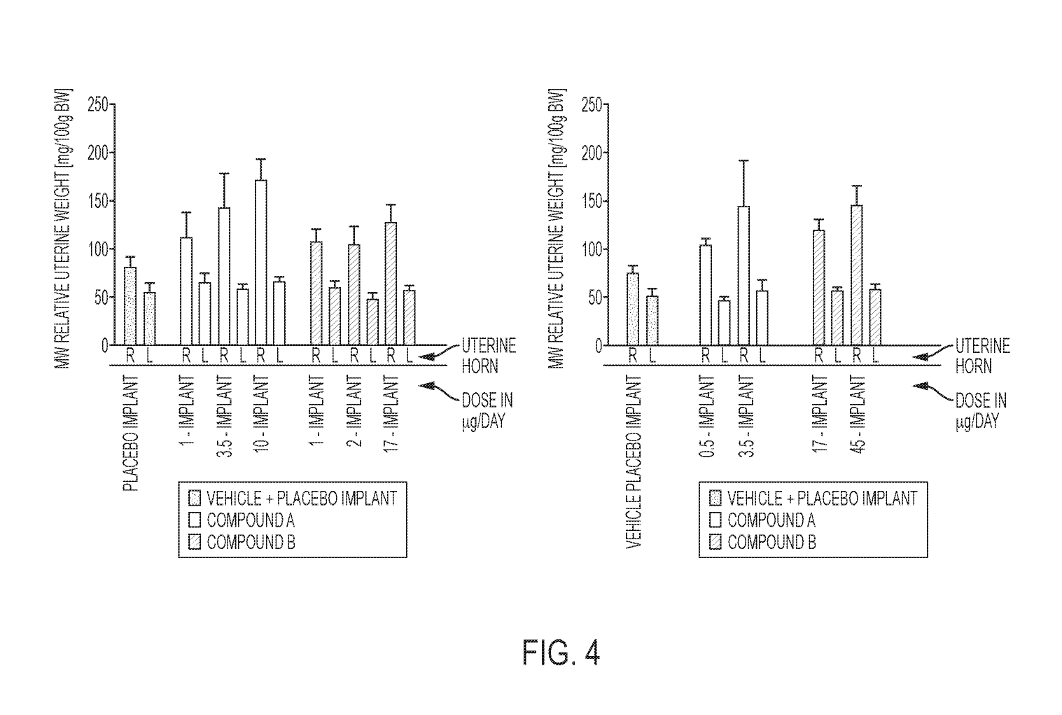

The local uterine action of the a.m. progestin compared to systemic side effects (dissociation) was investigated on the basis of studies using rats (see Example 4; FIGS. 4 to 6). The uterus of ovary-resected rats responded to implantation of progestin-containing IUS (rods) with decidualization and weight gain. The local progestin effects were also determined on the basis of changes in gene expression. The results of this experiment clearly show that 18-methyl-15β,16β-methylene-19-nor-20-spirox-4-en-3-one respectively its isomer can be dosed with local efficacy in such a way that the (systemic) side effects described for levonorgestrel do not occur in the woman.

The examples below serve to further illustrate the invention.

### EXAMPLE 1

### Core Preparation

65 parts by weight of 18-methyl-15β,16β-methylene-19-nor-20-spirox-4-en-3-one and 35 parts by weight of poly(dimethylsiloxane) elastomer were mixed in a closed mixer. The poly(dimethylsiloxane) elastomer used in the drug reservoir part is a silicon based fillerless PDMS (dimethylvinyl terminated poly[dimethyl-co-methylvinyl] siloxane) material which is crosslinked by hydrosilylation reaction by using platinum as a catalyst and poly(dimethyl-co-methylhydrogensiloxane) as a crosslinker. The drug containing mixture was extruded to a tube-like form with a wall thickness 0.8 mm and outer diameter of 2.8 mm and cured by heat during which crosslinking took place. The crosslinked core was cut into 5 and 8 mm lengths.

### Membrane Preparation for “Lower Release” Part (Reservoir 2)

The elastomer used in the membrane is a blend of two silica filler containing polysiloxane elastomers, PDMS (dimethylvinyl terminated poly[dimethyl-co-methylvinyl] siloxane and PTFPMS (poly(trifluoropropylmethyl-co-methylvinylsiloxane) elastomer, and crosslinked by hydrosilylation reaction by using platinum as a catalyst and poly(dimethyl-co-methylhydrogensiloxane) as crosslinker. PTFPMS is used in the membrane in combination with PDMS in ratio of 80/20 (PTFPMS/PDMS) to adjust the release rate of the drug substance.

### Membrane Preparation for “Higher Release” Part (Reservoir 1)

The elastomer used in the membrane is a silica filler containing polysiloxane elastomers, PDMS (dimethylvinyl terminated poly[dimethyl-co-methylvinyl] siloxane crosslinked by hydrosilylation reaction by using platinum as a catalyst and poly(dimethyl-co-methylhydrogensiloxane) as crosslinker.

The IUS consists of two separate parts of hormone-elastomer reservoir matrix mounted on a polyethylene T-body. Lengths of the parts are 5 and 8 mm. The membrane, consisting of a PTFPMS/PDMS blend with ratio 80/20, surrounds the drug core of length 8 mm and acts as a lower drug release rate part (wall thickness approx. 0.30 mm). The membrane, consisting of PDMS only, surrounds the drug core of length 5 mm (wall thickness approx. 0.4 mm).

The drug release rate level is predominantly controlled by the diffusion and partitioning (solubility) of the drug in the elastomer material, by the drug reservoir total surface area, and the membrane PTFPMS-content and membrane-thickness.

### EXAMPLE 2

### Drug Release Test

### Method

The release rate of the drug from the IUS was measured in vitro as follows:

The intrauterine delivery systems were attached into a stainless steel holder in vertical position and the holders with the devices were placed into glass bottles containing 75 ml of a dissolution medium. The glass bottles were shaken in a shaking water bath at 37° C. with 70 strokes/min. The dissolution medium was withdrawn and replaced by a fresh dissolution medium at predetermined time intervals, and the amount of the release drug was analyzed by using standard HPLC methods. The concentration of the dissolution medium and the moment of change (withdrawal and replacement) of medium were selected so that sink-conditions were maintained during the test.

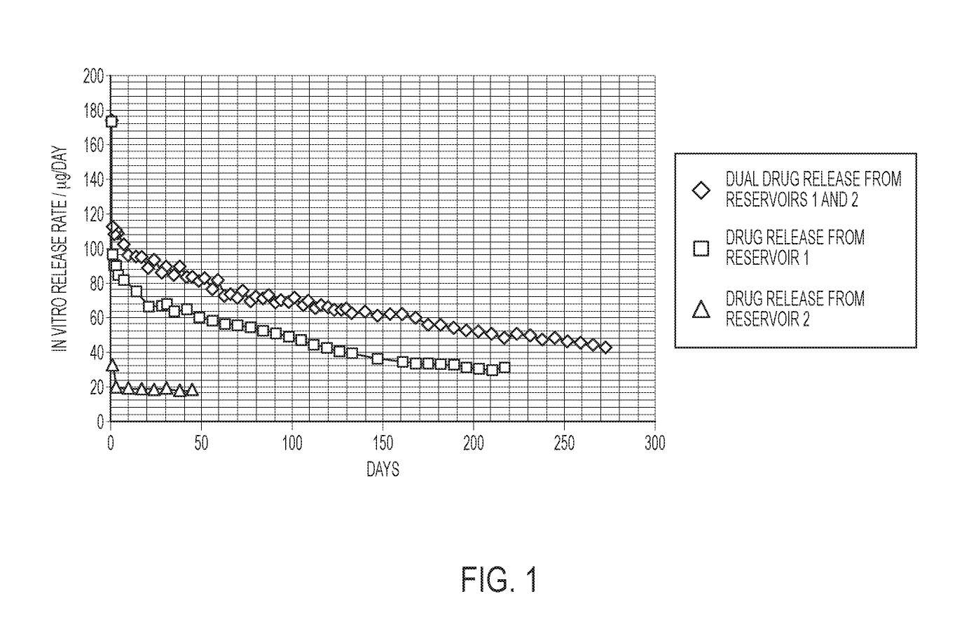

Results: The release rate obtained from separate parts and the combined system is illustrated in FIG. 1. As can be seen, the release rate from the pure PDMS membrane containing reservoir is higher and declines much faster for the first 3 to 6 months of treatment in this experiment and even up to 7-10 months. The release rate is constant for the IUS having a PTFPMS modified membrane and is known from previous experiments to continue steadily for a long period of time.

Systemically caused side effects, such as those occurring with the use of other gestagens, may thus be prevented or at least greatly reduced. Owing to the possible higher local gestagen concentration, a more rapidly commencing and better bleeding control can also be expected.

As a result, these progestins can be dosed with local efficacy in such a way that the side effects described for levonorgestrel do not occur in the woman.

### EXAMPLE 3

### Comparison Study in Monkey—Vehicle vs 2; 5 μg/d LNG vs 2; 5 μg/d NP

### Method

Animal Treatments: Adult cycling cynomolgus macaques were monitored to record regular menstrual cycles. Uterine bleeding was assessed daily by vaginal swabs (for sporadic vaginal spotting) and menstrual blood loss by vaginal tampons. After 2 menstrual cycles (˜60 days), the animals were assigned to treatment groups and laparotomized between days 6-8 (ideally day 7) of the follicular phase and an IUS was inserted by hysterotomy into the uterine lumen and sutured in place. Treatment IUS was as follows (n=5-6/group):
Group 1: Vehicle IUS
Group 2: 2 μg/day LNG
Group 3: 5 μg/day LNG
Group 4: 2 μg/day NP
Group 5: 5 μg/day NP

Classification of bleeddings: Bleedings were grouped in three categories: a) positive swap or frank menses, which is the most heavy form of bleeding (BB, red colour), b) light positive swab, which is an intermediated type of bleeding (B, purple colour) and c) spot positive swab, which is a very light form of bleeding (S, orange colour).

For evaluation of bleeding days, the first 7 days after insertion of the IUS were neglected, because the surgical insertion procedure already causes some bleedings in these days, which is unrelated progestin effects. FIGS. 2 and 3 and Tables 1 and 2 illustrate a comparison of 2 μg/d LNG release with 5 μg/d LNG release for 80 days with the IUS for each animal.

Results: The bleeding results for each animal are given in FIG. 2. The vehicle

IUS group showed cyclic bleeding patterns as expected for natural cycling animals. The 2 μg/d LNG release group showed a mixed pattern of bleeding in individuals, but on average less bleedings than the vehicle group. In contrast, markedly less bleeding was observed in the 5 μg/d LNG release group with marked reductions in all bleeding categories. (FIG. 2). A summarized comparison of the 2 and 5 μg/d LNG release groups is given in FIG. 3 Table 1.

New Progestin resulted in both release groups (2 and 5 μg/d) in a marked reduction of bleeding compared to the vehicle group. Comparison of bleeding for 2 μg/d LNG versus 2 μg/d NP clearly shows that NP leads to higher bleeding reduction than the same 2 μg/d LNG release (FIGS. 2 and 3, Tables 1 and 2) and therefore has a higher potency to reduce bleeding.

The results clearly show that a higher release rate of LNG results in a markedly reduced bleeding in the first months after IUS insertions in cynomolgus monkeys, which have a natural cycle and bleeding pattern very similar to women.

New Progestin also markedly reduces bleedings in the two tested release groups. Moreover, NP is a progestin with an even higher potency to reduce bleeding compared to LNG as seen by the comparison of the 2 μg/d release groups for both progestins.

Therefore a higher initial LNG or NP release could reduce or avoid the initial high bleeding burden known for Mirena® in the first months after IUS insertion⁸. ⁸ Andersson et al. Contraception 1994, 49:56-71

### EXAMPLE 4

Serum levels of luteinizing hormone (LH) are used for detecting systemic effects of the locally administered progestin. Basal serum-LH levels of ovary-resected rats are elevated compared to the levels of intact control animals. Undesired systemic efficacy of the uterine-administered progestin can be detected by a decrease in the LH level.

Ovary-resected female rats were treated with estradiol (E2) for three days (0.2 μg/day/animal, subcutaneous dosing). On day 4, an IUS (rod) was implanted into the right uterine horn of each animal. The left uterine horn remained untreated for internal comparison. Administration of E2 was continued with a daily dose of 0.1 μg/animal to ensure responsiveness of the uterus (maintaining progesterone-receptor expression) to progestins. Blood was taken for LH level measurements on days 4, 10 and 17.

### Performing the Gene Expression Analyses

The uterine tissue was homogenized in 800 μl of RLT lysis buffer (Qiagen, Hilden, Germany; #79216) using a Precellys24 homogenizer (Peqlab, Erlangen, Germany; 2.8 mm ceramic beads; #91-PCS-CK28, 2×6000 rpm). 400 μl of the homogenate obtained were used for isolating total RNA, using the QlAsymphony RNA kit (Qiagen, #931636) on a QlAsymphony SP robot for automated sample preparation. Reverse transcription of from 1 μg to 4 μg of total RNA was carried out using the SuperScript III first-strand synthesis system (Invitrogen, Carlsbad, USA; #18080-051) according to the random hexamer procedure.

Gene expression analysis was carried out with from 50 ng to 200 ng of cDNA per reaction on an SDS7900HT Real.time PCR system (Applied Biosystems, Carlsbad, USA) using TaqMan probes (Applied Biosystems; IGFBP-1 Rn00565713_m1, Cyp26a1 Rn00590308_m1, PPIA Rn00690933_m1) and the Fast Blue qPCR MasterMix Plus (Eurogentec, Liege, Belgium; #RT-QP2X-03+FB). For relative quantification, cyclophilin A (PPIA) was used as an endogenous control. Relative expression levels were calculated according to the comparative delta delta CT method.

### Results

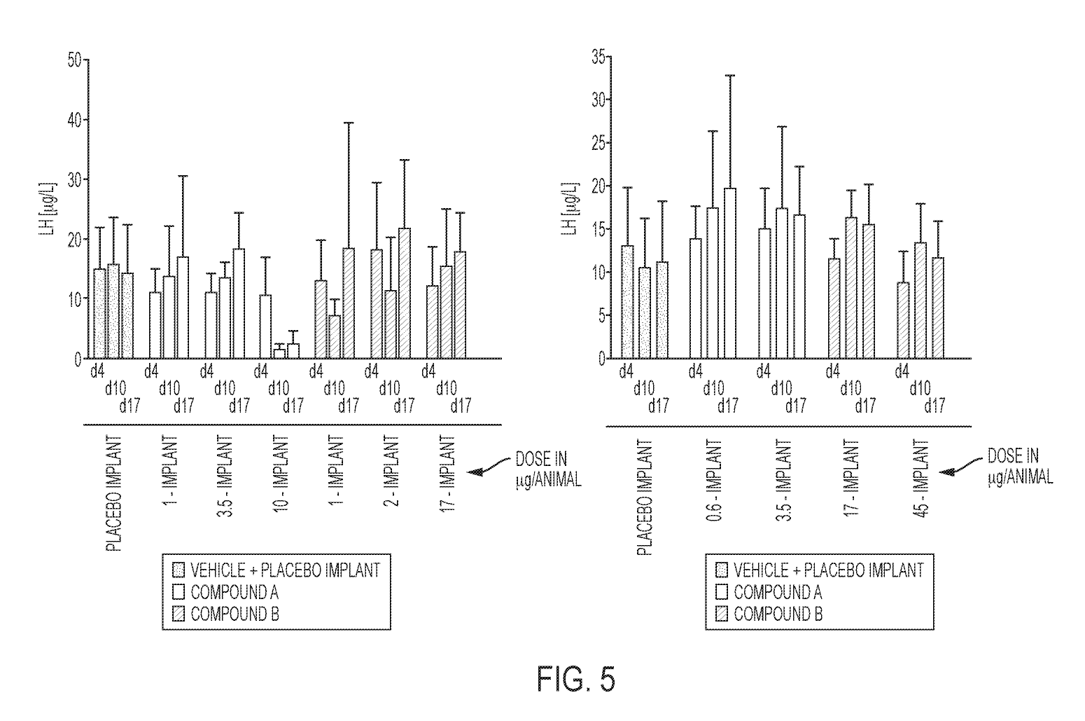

18-Methyl-15β,16β-methylene-19-nor-20-spirox-4-en-3-one (compound A) and 18-methyl-6α,7α,15β,16β-dimethylene-19-nor-20-spirox-4-en-3-one (compound B) exhibited dose-dependent local efficacy by way of weight gain in the IUS-carrying uterine horn (FIG. 5/7).

Within the release range tested (for compound A: 0.6-10 μg per animal and day, and for compound B: 1-45 μg/animal and day) both progestins surprisingly exhibited no LH decrease and therefore no systemic side effect, with the exception of the 10 μg/animal and day dose of compound A (FIG. 5).

The pharmacokinetic profile of 18-methyl-15β,16β-methylene-19-nor-20-spirox-4-en-3-one and 18-methyl-6α,7α,15β,16β-dimethylene-19-nor-20-spirox-4-en-3-one, respectively, indicated a very fast break-down rate in all in-vitro metabolic studies (liver) as well as in all animal species studied in vivo.

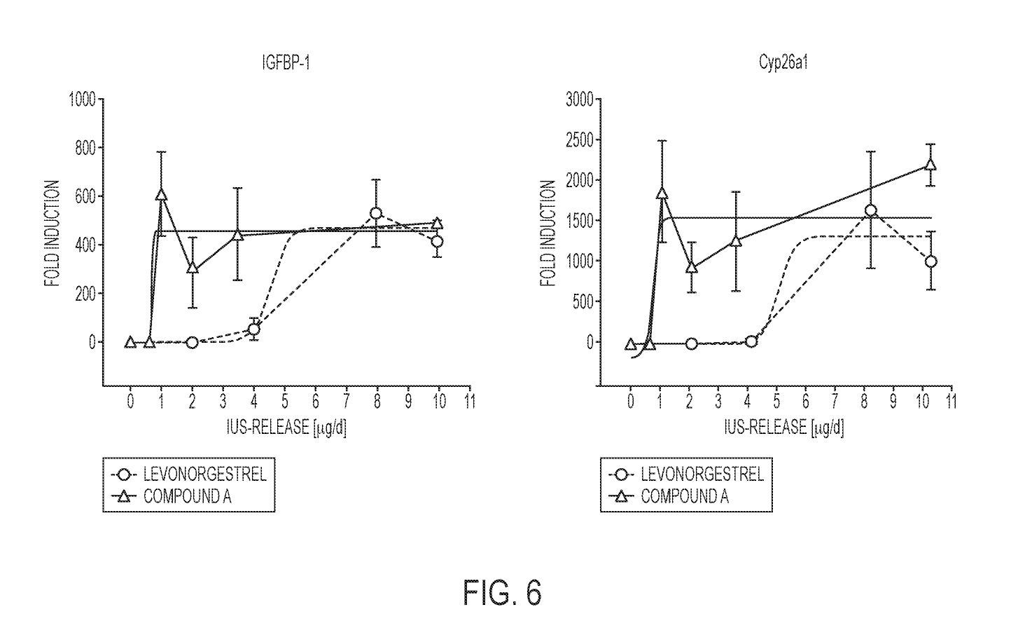

With local administration by means of an IUS (rods) in rats, compound A exhibited a 4- to 7-fold higher potency in inducing gene expression than levonorgestrel, with identical release rates (FIG. 6). This higher local potency additionally supports the possibility of achieving more rapid and stronger local gestagenic effects on the uterus without causing systemic side effects in the process.

1. An intrauterine delivery system comprising:
a body construction; and
two reservoirs (1, 2) comprising a core and a membrane encasing the core, the core and membrane consisting of a same or different polymer composition, wherein the reservoirs comprise progestogen or a drug having progestogenic activity which is released over a prolonged period time at a level required for contraception, characterized in that the release rates and times of release from the reservoirs (1,2) are different.

2. The intrauterine delivery system according to claim 1, characterized in that the membrane encasing the core of reservoir 1 essentially consists of polydimethylsiloxane (PDMS) or an elastomeric mixture of polyethylene oxide block-polydimethylsiloxane (PEO-b-PDMS) and PDMS or an elastomeric micture of polytrifluoropropylmethylsiloxane (PTFPMS) and PDMS.

3. The intrauterine delivery system according to claim 2, characterized in that the PEO-b-PDMS/PDMS ratio in the membrane of reservoir 1 is in the range of 95/5-0/100 (wt %).

4. The intrauterine delivery system according to claim 2, characterized in that the PTFPMS/PDMS ratio in the membrane of reservoir 1 is in the range of 20/80-0/100 (wt %).

5. The intrauterine delivery system according to claim 2, characterized in that the thickness of the membrane encasing the core of reservoir 1 is 0.15 to 0.3 mm.

6. The intrauterine delivery system according to claim 1, characterized in that the core of reservoir 1 is a tube-like form with an outer diameter of 2.5-3.0 mm and with a length of 4-16 mm.

7. The intrauterine delivery system according to claim 1, characterized in that the membrane encasing the core of reservoir 2 comprises a mixture of polytrifluoropropylmethylsiloxanes (PTFPMS) and PDMS.

8. The intrauterine delivery system according to claim 6, characterized in that the PTFPMS/PDMS ratio in the membrane of reservoir 2 is 100/0-10/90 (wt %).

9. The intrauterine delivery system according to claim 6 or 7, characterized in that the thickness of the membrane encasing the core of reservoir 2 is 0.3 to 0.6 mm.

10. The intrauterine delivery system according to claim 1, characterized in that the core of reservoir 2 is a tube-like form with an outer diameter of 2.5-3.0 mm, and with a length of 4-16 mm.

11. The intrauterine delivery system according to claim 1, characterized in that the membrane material contains silica filler, the content of which is used to further control the release rate.

12. The intrauterine delivery system according to claim 1, characterized in that the membrane consists of two or more layers, wherein the two or more layers comprise different membrane materials.

13. The intrauterine delivery system according to claim 1, characterized in that the amount of progestogen incorporated in the cores of reservoirs 1 and 2 is 45-55%, based on the weight of the core.

14. The intrauterine delivery system according to claim 1, characterized in that the progestogenic compound in both reservoirs is 18-methyl-15β,16β-methylene-19-nor-20-spirox-4-en-3-one.

15. The intrauterine delivery system according to claim 14, characterized in that the release rate of 18-methyl-15β,16β-methylene-19-nor-20-spirox-4-en-3-one from reservoir 1 is 10-200 μg/d for a period of at least three months.

16. The intrauterine delivery system according to claim 14, characterized in that the release rate of 18-methyl-15β,16β-methylene-19-nor-20-spirox-4-en-3-one from reservoir 2 is 1-20 μg/d for a period of at least three years.

17. The intrauterine delivery system according to claim 1, characterized in that the progestogenic compound in both reservoirs is levonorgestrel.

18. The intrauterine delivery system according to claim 17, characterized in that the release rate of Levonorgestrel from reservoir 1 is 20-100 μg/d, preferably 20-50 μg/d, for a period of at least three months.

19. The intrauterine delivery system according to claim 17, characterized in that the release rate of Levonorgestrel from reservoir 2 is 5-30 μg/d for a period of at least three years.

20. An improved method of contraception and for preventing or suppressing initial bleeding and spotting associated with the use of intrauterine delivery systems, wherein an intrauterine delivery system is used for the controlled release of progestogen or a drug having progestogenic activity over a prolonged period of time and at a level required for contraception and wherein said intrauterine delivery system comprises:
a body construction, and
two reservoirs comprising a core and a membrane encasing the core, the core and membrane consisting of a same or different polymer composition, characterized in that the release rates and times of release from the reservoirs are different.
